(19) United States
(12) Patent Application Publication
(10) Pub. No.: US 2002/0110602 A1
(43) Pub. Date: Aug. 15, 2002

(54) Method and apparatus for washing produce

(76) Inventor: David Sorenson, Lindsay, CA (US)

Correspondence Address: Sheppard, Mullin, Richter & Hampton LLP, 48th Floor, 333 South Hope Street, Los Angeles, CA, 90071, US

(21) Appl. No.: 09/932,432

(22) Filed: Aug. 17, 2001

Related U.S. Application Data
(60) Provisional application No. 60/226,270, filed on Aug. 17, 2000.

(51) Int. Cl. A61K 33/00; A61K 33/18

(52) U.S. Cl. 424/666; 424/600; 424/717

(57) ABSTRACT

A method for washing produce to inhibit or eliminate decay due to pathogens comprises washing the produce using a solution of alkaline salt of bicarbonate under conditions effective to inhibit or eliminate produce decay. Preferred embodiments include spraying the produce under pressure with a solution of alkaline salt of bicarbonate under preferred conditions, and immersing the produce in a solution of alkaline salt of bicarbonate and a sanitizing agent. Also, an apparatus for preventing or eliminating produce decay comprises components to facilitate use of the method of washing produce.

This application claims priority from U.S. Provisional Application Serial No. 60/226,270, filed Aug. 17, 2000.

### BACKGROUND OF THE INVENTION

This invention relates generally to a method for cleaning produce and, more particularly, to a method for cleaning produce to eliminate or reduce post-harvest pathogens and prevent the resulting damage. This invention also relates to an apparatus for cleaning produce using the method.

To accommodate market needs for produce, the produce needs to be preserved for extended periods of time in, for example, refrigerated storage for transit to distant markets. Whenever the period of time between harvesting and marketing of the produce is prolonged, however, the post-harvest decay caused by pathogens becomes exacerbated. For example, an incidence of rotted fruit of about 5 to 9%, or about 5 fruit per carton, is not uncommon. These fruit must be manually removed and, in addition to the loss produced by this procedure and labor requirements, often at shipper expense and in distant locations, the surviving fruit must be cleaned as well. Over time, this results in evident quality problems, causing foreign fruit buyers to seek other sources for the products.

Green mold, caused by Penicillium digitatum (Pers.:Fr.) Sacc. (P. digitatum), is one of the most economically devastating post-harvest diseases of citrus worldwide. The primary infections are wounds of P. digitatum on fruit inflicted during harvest and subsequent handling. These infections must be eradicated to achieve acceptable product quality. Currently, green mold is controlled in the U.S. by applying the fungicides imazalil, sodium ortho-phenyl phenate (“SOPP”), and thiabendazole (“TBZ”) to the fruit. About half of citrus packing houses have soak tanks, and the other half uses brush beds with chemicals to eliminate decay. The packing houses using brush beds currently generally utilize SOPP as a sanitizer and as a primary tool for decay control. Almost all packing houses also have high pressure scale washer (“HPSW”) facilities, which are primarily used for descaling citrus fruits and for general cleaning purposes. Post-harvest high pressure washing cleans and removes scale and insects from citrus fruit. First used in California in 1990 and now used in most of the approximately 100 citrus packing houses in California, these devices consist of rows of closely-spaced nozzles that deliver high volumes of water (100-200 mL/second each) at high pressure (up to 500 pounds per square inch). Use of HPSW alone does not control citrus green mold.

After green mold, sour rot, caused by Geotrichum candidum, is the second most devastating post-harvest pathogen of citrus fruit in California. Currently, there exists no chemical means for controlling sour rot. Accordingly, this pathogen can have an extremely negative impact at packing houses, where fruit is stored for long periods of time due to, for example, marketing delays.

In recent years, the development of pathogen resistance to the chemicals discussed above has been observed. For example, it has been reported that greater than about 90% of the isolates from California citrus packing houses have been resistant to imazalil, and that most strains already are resistant to SOPP and TBZ, the only other fungicides available to treat the fruit. These pathogen-resistant isolates are able to rot fruit with impunity, even when that fruit is treated with all three of these fungicides in recommended amounts. Moreover, regulatory issues and public concerns have arisen over the health risks of ingesting fungicide residues, threatening the continued viability of fungicide use in the future.

Besides the fungicides discussed above, inorganic cleaners, such as sodium carbonate (i.e., soda ash), borax/boric acid, and sodium bicarbonate, are of varying effectiveness in preventing fruit decay from green mold formation and other pathogens. Use of these inorganic cleaners does not lead to formation of resistance strains in the pathogens treated. In particular, sodium bicarbonates and carbonates are recognized as safe by the U.S. Food and Drug Administration and are common food additives for leavening, pH-control, taste, texture modification, and controlling spoilage. Brief immersion of citrus fruit in solutions of sodium bicarbonate or sodium carbonate reduces the subsequent incidence of post-harvest green mold caused by P. digitatum. Carbonates and bicarbonates work to loosen pathogenic spores from the surface of the fruit.

Sodium carbonate often is used in soak tank treatments and has particularly good eradicant activity when used to treat produce. For example, immersion in sodium carbonate reduces the incidence of infections from wounds on lemons inoculated 48 hours before treatment by more than 90%. The eradicant activity of a chemical is important, because most infections occur through wounds inflicted during or just after harvest, and often a day or more can elapse before treatments are applied. Sodium carbonate, however, is substantially insoluble and difficult to handle, particularly when used as part of a pressure washing treatment process. Sodium carbonate solutions also have a very high sodium content (twice that of sodium bicarbonate), and high pH. High sodium solutions can violate restrictions on discharge of such solutions. Treatment with a solution having pH higher than 9.5 generally is harmful to produce, leading to a more prevalent brown appearance of wounds on the fruit. High pH also can lead to difficulties in meeting regulations on discharges, requiring that the discharge be treated with acid to lower the pH before discharge. Therefore, use of soda ash generally is avoided, with the exception of its use in soaking tanks, where no viable alternative exists.

Use of sodium bicarbonate is inexpensive and poses a minimal risk of injury to the fruit. Sodium bicarbonate also can be a useful tool in the management of fungicide-resistant isolates, which have become particularly problematic. Its effectiveness approaches that of known fingicides employed for this purpose, and it is generally superior to other treatments that are alternatives to fungicides, such as heat or biological control. However, current uses of sodium bicarbonate for washing fruit for decay control in, for example, soak tanks, have not demonstrated a desirable level of effectiveness.

As discussed above, use of sodium bicarbonate for soaking produce is known to be effective for preventing decay from pathogens. Use of sodium bicarbonate also has been discussed in issued U.S. patents. For example, U.S. Pat. No. 1,098,006 to Allen discloses addition of hypochlorous acid to a vegetable pulp to render the pulp sterile, followed by addition of a carbonate of sodium to neutralize the acids before further preparation of the pulp. An example provided in the patent is that of mixing sodium hypochlorite with sodium bicarbonate, which produces sodium chloride. The disclosed method, however, is not suitable for treating whole fruit and does not involve washing the treatment substances off the fruit. This method is clearly not intended for washing the produce's surface, but rather to preserve pulp. U.S. Pat. No. 4,599,233 to Misato et al. discloses a composition utilizing sodium bicarbonate, a food emulsifier, and a carrier that is added to the produce for storage purposes, not for washing the produce. Neither of these two patents discloses a method for washing produce to prevent decay.

Accordingly, there exists a need for an effective method for preventing decay from pathogens of produce, particularly fruit, that is cost-effective and safe for routine use. The method should easily be implemented by packing houses without the need for costly new machinery. The present invention fulfills this need and provides further advantages.

### SUMMARY OF THE INVENTION

The present invention resides in a method for washing produce using washing solutions comprising an alkaline salt of bicarbonate under conditions for effective washing. The produce washed preferably is selected from the group consisting of oranges, lemons, tangerines, tangelos, grape fruits, grapes, bananas, apples, pears, peaches, nectarines, pomegranates, papaya, plums, melons, cucumbers, zucchini, carrots, mushrooms, peppers, broccoli, artichokes, cauliflower, tomatoes, potatoes, squash and celery.

An aspect of the present invention resides in a method for washing produce comprising spraying the produce using a washing solution under pressure for a duration, wherein the washing solution comprises an alkaline salt of bicarbonate and has a temperature and pH effective to inhibit or eliminate decay of the produce. The preferred alkaline salt of bicarbonate is sodium bicarbonate. The pressure of the washing solution preferably is between about 50 and about 500 lbs/in², more preferably between about 60 and about 350 lbs/in². The pH of the washing solution preferably is between about 7.0 and about 9.5, and more preferably between about 8.0 and about 8.4. The alkaline salt of bicarbonate preferably is present in an amount between about 0.25% and about 6% in the washing solution, more preferably between about 0.1% and about 5% in the washing solution, and most preferably in an amount of about 3% in the washing solution. The temperature of the washing solution preferably is between about 10° C. and about 40° C., and more preferably between about 20° C. and about 30° C. The duration of the spraying preferably is between about 1 second and about 10 minutes, more preferably between about 5 seconds and about 5 minutes, and most preferably between about 15 seconds and about 1 minute. The washing solution may comprise a sanitizing agent, preferably selected from the group consisting of ClO₂, ozone, and an alkaline salt of hypochlorite. The preferred sanitizing agent is sodium hypochlorite present in the washing solution at a concentration of about 200 ppm. The method may further comprise a step of immersing the produce in a second solution comprising an alkaline salt of bicarbonate. The method also may further comprise a step of rinsing the produce using an aqueous solution after the step of spraying the produce.

Another aspect of the invention resides in a method for washing produce comprising contacting the produce with a washing solution comprising an alkaline salt of bicarbonate and a sanitizing agent at a concentration, for a duration, and at a temperature and pH effective to inhibit or eliminate decay of the produce. This contacting can be in the form of immersing the produce in the washing solution or spraying it with the solution. The sanitizing agent preferably is selected from the group consisting of ClO₂, ozone, and an alkaline salt of hypochlorite. The preferred sanitizing agent is sodium hypochlorite present in the washing solution at a concentration of about 200 ppm. The preferred bicarbonate species, bicarbonate concentration, pH, temperature, and duration are as was described above. The method preferably also comprises a step of rinsing the produce in an aqueous solution after the step of contacting the produce.

Another aspect of the invention resides in an apparatus for use in washing produce, comprising an alkaline salt of bicarbonate, a sanitizing agent, and instructions for employing the alkaline salt of bicarbonate and sanitizing agent in washing produce under conditions effective in inhibiting or eliminating decay of the produce. The apparatus can further comprising a measuring means or an agent for adjusting the pH of a washing solution prepared using the apparatus. The alkaline salt of bicarbonate preferably is sodium bicarbonate. The sanitizing agent preferably is selected from the group consisting of ClO₂, ozone and an alkaline salt of hypochlorite.

Other features and advantages of the present invention should become apparent from the following detailed description of the invention, taken with the accompanying figures, which illustrate the principles of the invention.

### BRIEF DESCRIPTION OF THE DRAWINGS

FIG. 1 is a graphical representation of the percentage decay control on oranges under the following treatments: (a) Inoculation only; (b) Pressure washing with 200 ppm HOCl; (c) Pressure washing with SOPP spray followed by high-pressure washing with 200 ppm HOCl; (d) Dipping with sodium bicarbonate plus 200 ppm HOCl; and (e) Pressure washing with sodium bicarbonate plus 200 ppm HOCl.

FIG. 2 is a graphical representation of green mold incidence in oranges and lemons that were injected with green mold under the following treatments: (a) Inoculation only; (b) Pressure washing with borax/boric acid; (c) Dipping in sodium bicarbonate (d) Pressure washing in sodium bicarbonate; and (e) Dipping in soda ash.

FIG. 3A is a graphical representation of green mold incidence in oranges under the following treatments: (a) Inoculation only; (b) Dipping in 3% sodium bicarbonate; (c) Pressure washing in 3% sodium bicarbonate; (d) Dipping in Boric acid/borax; (e) Pressure washing in Borax/boric acid; and (f) Dipping in 3% soda ash.

FIG. 3B is a graphical representation of green mold incidence in lemons under the following treatments: (a) Inoculation only; (b) Dipping in 3% sodium bicarbonate; (c) Pressure washing in 3% sodium bicarbonate; (d) Dipping in Boric acid/borax; (e) Pressure washing in Borax/boric acid; and (f) Dipping in 3% soda ash.

FIG. 4A is a graphical representation of green mold incidence in lemons under the following treatments: (a) Inoculation only; (b) Dipping in 3% Sodium bicarbonate; and (c) Pressure washing in 3% Sodium bicarbonate.

FIG. 4B is a graphical representation of green mold incidence in lemons under the following treatments: (a) Inoculation only; (b) Dipping in sodium bicarbonate; and (c) Pressure washing in sodium bicarbonate.

FIG. 5 is a graphical representation of the percentage decay control on oranges under the following treatments: (a) Washing with solution having no HOCl at about 400 psi; (b) Washing with solution having high HOCl at about 400 psi; (c) Washing with solution having no HOCl at about 400 psi; and (d) Washing with solution having high HOCl at about 400 psi.

FIG. 6 is a graphical representation of results on decay control on oranges and lemons by pressure washing using varying concentrations of sodium bicarbonate: (a) 0.5%; (b) 1%; (c) 3%; and (d) 3%, in which lemons used are three days old.

FIG. 7 is a graphical representation of results on fruit decay control of dipping using varying concentrations of sodium bicarbonate: 0.25%; 0.5%; 1.0%; and 3%.

### DESCRIPTION OF THE PREFERRED EMBODIMENTS

The present invention is embodied in a method for reducing or eliminating the post-harvest decay of produce from pathogens by washing of the produce using a solution of an alkaline salt of bicarbonate of a concentration, under conditions effective to reduce or eliminate such decay. The present invention also is embodied in an apparatus for use in washing of produce using the described method.

In a preferred embodiment of the invention, the produce is washed under using a high-pressure spray of a solution containing an alkaline salt of bicarbonate. The alkaline salt of bicarbonate used in the solution preferably is sodium bicarbonate, because of the relatively high efficacy of the sodium bicarbonate and the relatively low cost. Also, for example, potassium bicarbonate or potassium carbonate can be used. The washing pressure can be any pressure above ambient pressure. Preferably, the washing pressure ranges from about 50 to about 500 lbs/in², and more preferably from about 60 to about 350 lbs/in². In the preferred embodiment of the invention, sodium bicarbonate used is at a concentration ranging from about 0.25% to about 6.0% by weight, more preferably from about 0.1% to about 5.0% by volume, and most preferably about 3.0% by weight. Lower concentrations of sodium bicarbonate generally provide lower efficacy of decay control. The pressure washing preferably is conducted for a period of time ranging from about 1 second to about 10 minutes, and more preferably from about 5 seconds to 5 minutes, most preferably about 15 seconds to 1 minute. The pressure washing may take place at any temperature that does not damage the produce. Preferably, the washing is conducted at a temperature of about 10° C. to about 40° C., and more preferably at about 20° C. to about 30° C. The pressure washing can be conducted at any pH that will not damage the produce being washed. In the preferred embodiment, the pH ranges from about 7.0 to about 9.5, and more preferably from about 8.0 to about 8.4.

In this embodiment of the invention, the solution used for pressure washing can incorporate a sanitizing agent to kill pathogens as well as remove them from the surface of the produce. Examples of sanitizing agents include ClO₂, ozone and an alkaline salt of hypochlorite. Sodium hypochlorite is the preferred sanitizing agent, because of its low cost, ready availability, and good sanitizing ability. Addition of sodium hypochlorite as a chlorine source enhances the solution's cleaning efficacy and provides additional fungicidal control. Preferably, the sodium hypochlorite is added to the solution to a level of 200 ppm. The method of the present invention allows for incorporation of chlorine washing far more effectively than other known methods for cleaning. Use of other tank solutions approved and in commercial use, including liquid lime sulfur solution, borax/boric acid mixtures, sodium carbonate, and SOPP, either are not compatible with chlorine or are only stable at extremely high pH. Chlorination efficiency is improved when the chlorine is applied at near-neutral pH, because a larger portion of the hypochlorite present is protonated and in the more active germicidal form as hypochlorous acid. Because the alkaline salt used in the present invention serves as a high-capacity buffer, it keeps the cleaning solution at an optimum pH for chlorination. This chlorination is effective in preventing microbial contamination of bicarbonate salt solutions, and to increase the efficacy of sodium bicarbonate, for example, in controlling citrus green mold. This effective incorporation of chlorine into the solution removes the need for sanitizing the produce via heating the solution, as is used for solutions incorporating sodium carbonate or borax. These solutions generally are heated to 105° C. to 110° C. to kill pathogens during treatment, leading to possible reduction in the quality of the produce treated.

Another embodiment of the method of the present invention includes a step of contacting the produce in washing solution incorporating an alkaline salt of bicarbonate, preferably sodium bicarbonate, and a sanitizing agent, preferably sodium hypochlorite. This contacting can be in the form of, for example, immersion of the produce for short time intervals as in a dip tank, or immersion in a soak tank for longer time intervals. Addition of sodium hypochlorite to the sodium bicarbonate provides for good treatment of produce, in contrast to known methods of immersion in sodium bicarbonate. The alkaline salt of bicarbonate and sanitizing agent work synergistically to improve treatment beyond use of either alone in solution. The sodium bicarbonate loosens the pathogens, such as spores, from the surfaces of the produce, and the chlorine kills the pathogens. This allows effective treatment, even when using immersion systems that incorporate recirculation of treating solution and subsequent build-up of pathogens in the solution. Because the sanitizing agent kills these pathogens, this build-up does not substantially reduce the treating efficacy of the solution.

The contacting preferably takes place using the same levels of concentration, temperature, and pH as the previously-described embodiment of the invention. The contacting preferably is conducted for a period of from about 1 second to about 10 minutes, and more preferably from about 5 seconds to 5 minutes. The contacting can be conducted in conjunction with the embodiment of treating with spray under pressure as has been described above.

Particularly when a solution incorporating a sanitizing agent having chlorine, such as sodium hypochlorite, is used, the method of the present invention preferably includes a step rinsing the produce with an aqueous solution after treating with the solution incorporating the alkaline salt of bicarbonate and the chlorine sanitizing agent. This rinse removes any chlorine residues from the treated produce. The rinse can be at any temperature that does not injure the produce, for a duration sufficient to rinse the produce of these residues.

The method of the present invention can be used in conjunction with a pressure washing system having a large screen filter installed to adequately filter the high pressure scale washer (HPSW) water. However, other known filtration means also may be utilized, such as a sand media filter. Use of a sand media filter, however, requires substantial backflushing, resulting in substantial loss of sodium bicarbonate in the backflush water. A metering device, such as a food ingredient auger, also may be installed for dispensing the sodium bicarbonate into the HPSW water. For high pressure washers, which operate at a range from about 70 to about 400 psi, sodium bicarbonate is present in the solution preferably in an amount of about 0.5% to about 3%. The solution is at a temperature of about 40° C. The solution is applied though a nozzle over a brushbed, and the efficacy of the method will vary depending on the pressure applied though the washer and the concentration of solution used to wash the fruit.

The method of the present invention also can be used with a “hot soak tank”-like treatment, using a HPSW and sodium bicarbonate on a commercial packing house brush bed. In this embodiment, the sodium bicarbonate is present in the washing solution preferably in a concentration of from about 0.5% to about 3%. Also, a concentration of about 200 ppm of sodium hypochlorite should be maintained in the solution for optimum treatment. The temperature of the tank should be maintained within a range of from ambient temperature to about 105° C. As the temperature increases within this range, efficacy of the solution also increases.

The present invention also includes an apparatus for preventing or eliminating produce decay, comprising the following components in separate containers: an alkaline salt of bicarbonate, preferably sodium bicarbonate; a sanitizing agent; and instructions for employing these components in the inhibition or elimination of produce decay consisted with the method of the present invention. The sanitizing agent provided with the kit may be, for example, ClO₂, ozone, hypochlorous acid, or an alkaline salt of hypochlorite. The apparatus also may comprise a measuring means for dispensing the ingredients, or the ingredients may be provided in unit form, preferably sealed to prevent contamination. The kit may further comprise one or more pH adjusting agents for use in adjusting the acidity of the washing solution produced.

Consistent application of the method of the present invention to post-harvest produce, and particularly citrus fruit, lowers microbial hazards to the fruit, and therefore the resulting decay. The method of the present invention provides equivalent or superior results when compared with conventional treatment methods, and it allows for reduced chemical exposure of the produce if desired. Use of the present invention requires only little or no costs for redesigning washers and filters commonly used in fruit processes to permit the handling of bicarbonate. The method also provides energy savings when used in place of a hot soak tank treatment, because it can utilized as part a cold treatment that does not require heating of the solution used. The method of the present invention can result in a significant reduction in the use of traditional fungicides in packing houses, reducing chemical exposure and chemical costs during packing, transportation, and storage of the produce. For example, use of present invention can reduce the amount of imazalil used on lemons or oranges going into short-term storage, as well as oranges. It can also be helpful in reducing the use of SOPP on the brush beds of citrus packing houses. Therefore, use of the present invention provides an environmentally friendlier option for controlling post-harvest decay than current methods.

The present method may be applied to all kinds of produce, including but not exclusively limited to, fruits and vegetables. The fruit may be any kind of fruit, preferably those having hard skins, such as citrus, e.g, oranges, lemons, tangerines, tangelos, grape fruits, and kumquats, as well as grapes, bananas, apples, pears, peaches, nectarines, pomegranates, papaya, plums, melons, watermelons, and many more. The method of the present invention also may be applied for preventing decay of any type of vegetables. However, the method works best with hard skin vegetables, such as cucumbers, zucchini, carrots, mushrooms, peppers, broccoli, artichokes, cauliflower, tomatoes, tubers such as potatoes, sweet potatoes, squash, etc., and celery, among others.

### EXAMPLES

Various experiments were performed to measure prevention of decay using various embodiments of the present invention in comparison to existing methods.

1. General Methodology

P. digitalum was commonly used to evaluate the effectiveness of the method of the present invention in comparison to conventional methods. A double screen filter was used to keep the HPSW contained, while the suitable HPSW containing the particular chemicals being used for each test was run through the filter. The sand media filters used, although acceptable, had to be backflushed three to four times a day, consuming about 1,000 gallons of water. A dry auger feeder was added to the system to meter the flow of bicarbonate and other chemicals. The water flows ranged from about 350 to about 600 gallons a minute in the HPSW. Agitation equipment was added.

Both naturally and artificially inoculated produce were exposed to P. digitatum. The day before each experiment, all fruit was randomized and inoculated with 1,000,000 spores/mL. In addition, petri dishes of potato dextrose agar were inoculated with various isolates of P. digitatum, and incubated at 20° C. for 1 to 2 weeks. Spores were rubbed from the agar with a glass rod in a small volume of sterile water containing 0.05% of Triton X-100. The spore suspension was passed through two cheese cloths and diluted with water to an absorbance of 0.1 at 420 nm. The density was approximately 1 million spores/mL, as recommended for evaluation of green mold. Each fruit was wounded and inoculated once by dipping a stainless steel rod in the spore solution and making a puncture 1 mm wide and 2 mm deep on each fruit. After inoculation, the fruit were incubated for about 24 hours at 15° C. to 20° C. The incidence of decay by these pathogens was determined, usually several weeks later, depending upon the temperature and maturity of the fruit tested.

2. Evaluation Criteria

The effectiveness of the decay control of green mold was compared using a high pressure scale washer and sodium bicarbonate solutions within the scope of the present invention to various methods of treatment. Also, the cost of using a cold sodium bicarbonate solution in the HPSW was compared versus the decay control cost of a heated soak tank. The use of a chemical in the pump system and filter systems was studied for any wear that may be of harm to packing house equipment. Finally, the quality of the water was evaluated after several days of use to determine its aseptic qualities, e.g., spores in solution, dirt load characteristics and how it affected use of the bicarbonate.

3. Effect on Decay Control of Various Methods of Treatment

Fruit inoculated as described above was incubated for 24 hours at 20° C. One hundred pieces of fruit, were employed per each replicate of study, and four replicates were conducted per each treatment. The inoculated fruit was used in the following treatments:

1) Control (untreated).

2) 3% Sodium bicarbonate solution in the HPSW (alone)

3) 3% Sodium bicarbonate solution in the HPSW/200 ppm HOCl.

4) 3% Sodium bicarbonate solution/200 ppm HOCl (dip).

5) 2% SOPP Brush bed spray treatment (conventional packing house treatment).

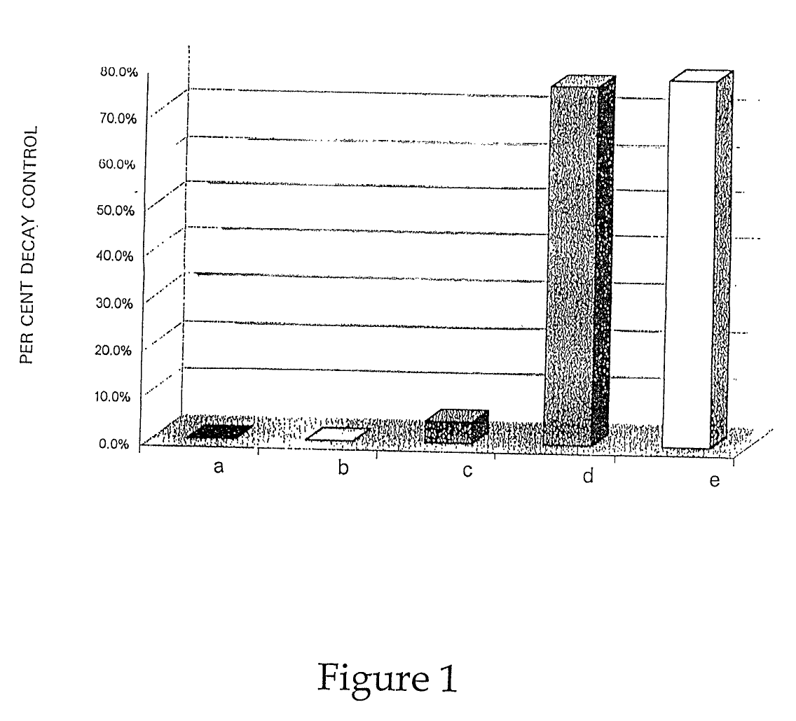

The data was subjected to statistical analysis, and the fruit treatments were analyzed after 2 weeks in storage at 45° F. Results, shown in FIG. 1 and discussed further below, indicated that the treatments within the scope of the present invention provided excellent decay control in the treated fruit.

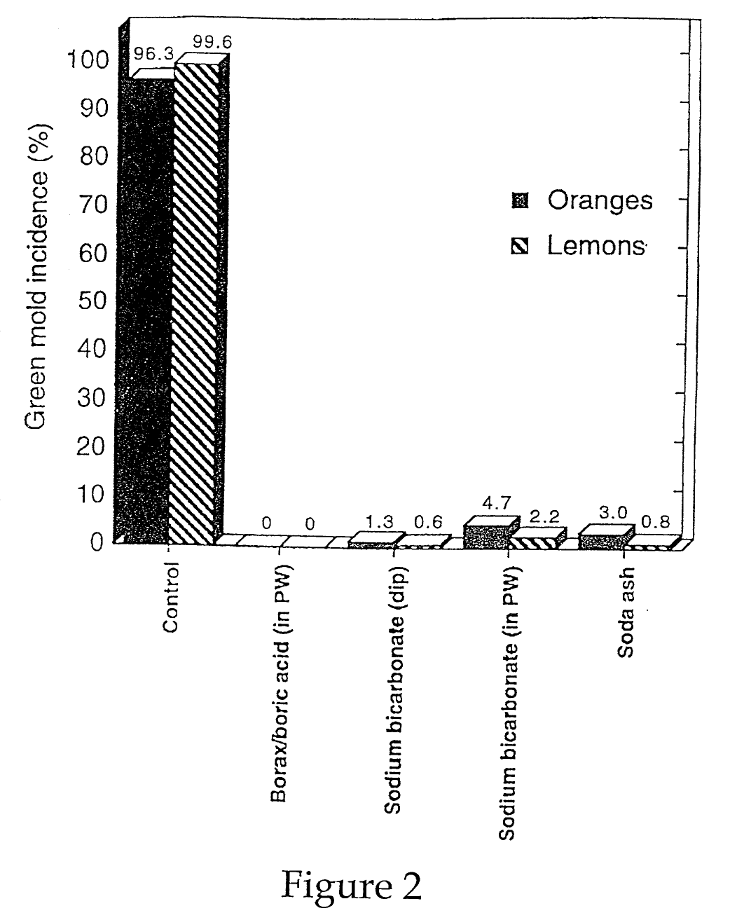

In a separate experiment, fruit tested was inoculated as described above. A solution within the scope of the present invention containing 3% sodium bicarbonate was tested and compared to a standard 3% soda ash solution, and a 4% borax/2% boric acid solution. As the HPSW was re-circulated to the tanks containing the solution, it became chlorinated to a level of about 50 ppm. Because the chlorine activity in sodium bicarbonate at a pH of 8.3 is less than that at a pH of 7, the concentration of chlorine was adjusted to 200 ppm. Results of this testing, shown in FIG. 2, indicate there is no difference between a soak tank treatment and HPSW treatment using sodium bicarbonate and chlorine.

4. Effect on Decay Using High Pressure Washing Outside the Scope of the Present Invention

The effect of high pressure washing with a solution containing sodium hypochlorite as a chlorine source on decay control of oranges were examined. An apparatus as described above was used for high pressure washing. No alkaline salt of bicarbonate was added to the solution. Navel oranges were collected from a dump elevator and inoculated to a depth of 3 mm with a solution containing 1,000,000 spores per mL of sensitive P. digitatum (M6R strain). The oranges was randomized into five different groups, 1 to 5, with each group containing about 40 oranges, for application of the five different treatments shown in Table 1 below. The oranges then sat covered overnight on the floor of the packing area. The oranges pulped at 56° F. on the first day, and 55° F. on the second day.

The next morning, oranges in Groups 2 to 5 were subjected to their respective treatments by passing them through the high pressure washer under the conditions described in Table 1, and the treated oranges was collected just after the brush bed. The pressure conditions used were as follows: “low pressure” was about 95 psi and “high pressure” was about 300 psi. The brushes were located approximately 6 inches from the washer nozzles. Although ortho phenyl phenate (OPP) was used as a standard treatment at the facility, the foamer was turned off and the brush bed rinsed. Therefore, no appreciable OPP residues were found in samples collected to affect the levels of decay in the samples.

The oranges then were incubated at 55° F. for two weeks and evaluated for decay. No significant differences in degree of decay were found at various pressure rates and chlorine concentrations. The results are shown in Table 1 below.

TABLE 1
Decay Control Using High Pressure Washing
Outside the Scope of the Invention

| | | | | % Fruit Decay |
|---|---|---|---|---|
| No. | Treatment Conditions | Total Decay | No decay | (Average) |
| 1 | Control⁺ | 200 | 2 | 99 |
| 2 | Low P Low Cl | 199 | 1 | 99.5 |
| 3 | High P Low Cl | 201 | 3 | 98.5 |
| 4 | Low P High Cl* | 196 | 4 | 98 |
| 5 | High P High Cl* | 197 | 5 | 97.5 |

P: Pressure; Cl: HOCl concentration
*Chlorine levels uncertain
⁺No high pressure washing

The results show that washing, even under pressure, using a solution of water and sodium hypochlorite had little or no effect on pathogen infection and fruit decay at varying concentrations of pressure and chlorine content.

5. Effect on Decay Using Immersion Washing With Various Solutions

A test was performed using four groups of 25 oranges exposed to treatment using the following aqueous solutions: 1) 0.25% sodium bicarbonate; 2) 0.5% sodium bicarbonate; 3) 1% sodium bicarbonate; 4) 3% sodium bicarbonate; 5) 0.25% sodium bicarbonate and 200 ppm sodium hypochlorite; 6) 0.5% sodium bicarbonate and 200 ppm sodium hypochlorite; 7) 1% sodium bicarbonate and 200 ppm sodium hypochlorite; 8) 3% sodium bicarbonate and 200 ppm sodium hypochlorite; 9) 1% sodium bicarbonate and 200 ppm sodium hypochlorite at elevated temperature; 10) 0.5% sodium bicarbonate and 200 ppm sodium hypochlorite at elevated temperature; 11) 200 ppm sodium hypochlorite; 12) Inoculation of fruit only and 13) water solution only (control). Four samples of 25 oranges each were counted, and the decay present in these oranges was expressed as the mean percentage for each of the four samples. The results of this experiment are shown in Table 2 below, with the treatments arranged in increasing order of decay observed.

TABLE 2
Decay Control Using Immersion With Various Solutions

| | Mean Decay |
|---|---|
| Treatment | (%) |
| BCB 3% + 200 ppm HOCl | 2.000 |
| BCB 3% alone | 7.000 |
| BCB 0.5% + 200 ppm HOCl | 9.000 |
| BCB 1% + 200 ppm HOCl | 9.000 |
| Heated BCB 1% + 200 ppm | 11.000 |
| HOCl | |
| Heated BCB 0.5% + | 11.000 |
| 200 ppm HOCl | |
| BCB 1% alone | 13.000 |
| BCB 0.5% alone | 27.000 |
| BCB 0.25% + 200 ppm | 30.000 |
| HOCl | |
| BCB 0.25% alone | 46.000 |
| HOCl 200 ppm alone | 88.000 |
| Water only (Control) | 97.000 |
| Inoculate (Control) | 97.000 |

The results show that the treatments within the scope of the present invention prevented between 54% and 98% decay of the fruit, depending upon the concentration of bicarbonate and the addition of optional chemicals. The decay-preventing effect of sodium bicarbonate increases with increased concentration of the compound. The addition of sodium hypochlorite also is advantageous. The samples treated with water only or subjected only to inoculation decayed almost completely, as did those treated with sodium hypochlorite alone.

6. Effect on Decay Using Washing of Oranges With Various Solutions on Green Mold

A procedure similar to that procedure as that discussed above in Part 5 was used, except for use of an inoculus of Green Mold. Four groups of 25 oranges each were inoculated as described above and treated as described with the compounds specified in Table 3 under the conditions described in Part 5 above. All of the oranges were treated using a pressure washer, except those treated with soda ash, sodium bicarbonate or borax/boric acid, which were treated using a soak tank. The control with inoculus alone was injected and allowed to stand. The results of decay on these oranges are shown in Table 3 below.

TABLE 3
Decay Control on Oranges Infected by Green Mold

| Treatment | Mean Decay (%) |
|---|---|
| Sodium bicarbonate 3% w/v Rinse + 200 ppm Oxine | 7.000 |
| (chlorine dioxide) at 80° F. | |
| Inoculate alone (Control) | 8.000 |
| Oxine 3-6 ppm + 200 ppm NaOCl pH 7.5 80° F. | 8.000 |
| Dequest 2016D Phosphonic acid 1.9% w/v pH 12 | 10.000 |
| (no rinse) | |
| Soda ash 3% w/v at 80° F. | 11.000 |
| Oxine 200 ppm + 200 ppm NaOCl pH 7.5 at 80° F. | 13.000 |
| Soda ash 3% wt/vol, rinse, 3-6 ppm Oxine at 80° F. | 13.000 |
| Oxine 75 ppm alone at 80° F. | 14.000 |
| Oxine 3-6 ppm alone at 80° F. | 14.000 |
| NaOCl 200 ppm pH 7.5 alone at 80° F. | 17.000 |
| Oxine 200 ppm alone at 80° F. | 18.000 |
| Inoculate in Water at 80° F. (Control) | 19.000 |
| Lime sulfur 3% w/v at 80° F. | 24.000 |

The results presented in Table 3 show that of the control methods use, use of sodium bicarbonate along with Oxine (i.e., ClO₂) as a sanitizer, produced the lowest level of decay. The least protection against decay in the methods used in was attained using a solution of lime sulfur.

7. Effect of pH and Bicarbonate Concentration on Decay Control

The following data correspond to two separate experiments conducted using separate groups of oranges. Four groups each of 25 oranges were subjected to each of the treatments specified in Table 4 to determine the effect on the method of the present invention of varying pH and concentration of sodium bicarbonate in the solution. The treatment of the oranges was performed as described in Part 1 above, and the results are shown in Tables 4 and 5 below.

TABLE 4
Effect of Varying pH Using Different Treatments on Fruit Decay
First Set of Data

| | Mean | Standard | Standard |
|---|---|---|---|
| Treatment | Decay (%) | Deviation | Error |
| 0.15 M sodium bicarbonate, pH 7.5 | 65.000 | 12.606 | 8.403 |
| 0.15 M sodium bicarbonate, pH | 63.000 | 10.520 | 5.260 |
| 7.5, HOCl 200 ppm | | | |
| 0.15 M soda ash, pH 11.4 | 31.000 | 11.469 | 6.745 |
| 0.15 M soda ash, pH 11.4, HOCl | 25.000 | 10.520 | 5.280 |
| 200 ppm | | | |
| 0.3 M sodium bicarbonate, pH 7.5 | 41.000 | 18.000 | 9.000 |
| 0.3 M sodium bicarbonate, pH 7.5 | 35.000 | 8.246 | 4.123 |
| HOCl, 200 ppm | | | |
| 0.3 M sodium bicarbonate, pH 8.4 | 43.000 | 11.489 | 5.745 |
| 0.3 M, sodium bicarbonate, pH | 32.000 | 7.303 | 3.651 |
| 8.4, HOCl 200 ppm | | | |
| 0.3 M soda ash, pH 11.4 | 10.000 | 4.000 | 2.000 |
| 0.3 M soda ash, pH 11.4, HOCl | 10.000 | 6.928 | 3.464 |
| 200 ppm | | | |
| Agriquest Bacillus OST 713 10 g/L | 99.000 | 2.000 | 1.000 |
| Agriquest Bacillus OST 713 20 g/L | 88.000 | 11.314 | 5.657 |
| HOCl pH 11.4 | 95.000 | 2.000 | 1.000 |
| HOCl pH 7.5 | 99.000 | 2.000 | 1.000 |
| HOCl pH 8.4 | 99.000 | 2.000 | 1.000 |
| Inoculus, water treated (Control) | 95.000 | 3.830 | 1.915 |
| Inoculus (Control) | 99.000 | 2.000 | 1.000 |
| Methyl jasmenate, high rate | 100.000 | 0.000 | 0.000 |
| Methyl jasmenate, low rate | 100.000 | 0.000 | 0.000 |

TABLE 5
Effect of Varying pH Using Different Treatments on Fruit Decay
Second Set of Data Dipping

| Treatment | Mean Decay (%) |
|---|---|
| 0.3 M soda ash, pH 11.4, HOCl | 10.000 |
| 200 ppm | |
| 0.3 M soda ash, pH 11.4, alone | 10.000 |
| 0.15 M soda ash, pH 11.4, HOCl | 25.000 |
| 200 ppm | |
| 0.15 M soda ash, pH 11.4, alone | 31.000 |
| 0.3 M Sodium bicarbonate, pH 8.4, | 32.000 |
| HOCl 200 ppm | |
| 0.3 M Sodium bicarbonate, pH 7.5 | 35.000 |
| HOCl 200 ppm | |
| 0.3 M Sodium bicarbonate, pH 7.5, | 41.000 |
| alone | |
| 0.3 M Sodium bicarbonate, pH 8.4, | 43.000 |
| alone | |
| 0.15 M Sodium bicarbonate, pH | 63.000 |
| 7.5, HOCl 200 ppm | |
| 0.15 M Sodium bicarbonate, pH | 65.000 |
| 7.5, alone | |
| Agriquest Bacillus QST 713 20 g/L | 88.000 |
| inoc., water treated control | 95.000 |
| HOCl, pH 11.4 alone | 95.000 |
| HOCl pH 7.5 alone | 99.000 |
| HOCl pH 8.4 alone | 99.000 |
| Agriquest Bacillus QST 713 10 g/L | 99.000 |
| inoculation (control) | 99.000 |
| Melthy jasmonate, high rate | 100.000 |
| Melthyl jasmonate, low rate | 100.000 |

+ HOCl effect, P = 0.12 orthogonal
Paired t test, P = 0.0635

The results shown in Tables 4 and 5 demonstrate that within the prescribed range, the higher concentrations of bicarbonate are substantially more effective than the lower ones (0.15%) for controlling fruit decay. In addition, the data show that within the range tested there is no significant change produced by pH changes and that whether alone or with sodium hypochlorite, sodium bicarbonate is effective in controlling decay when applied to fresh oranges.

8. Effect of Treatments on Sour Rot Decay

Sour rot is a typical pathogen for lemons. Two experiments were conducted to test the method of the present invention on the sour rot pathogen and the resulting decay on lemons. In each experiment, four groups of 25 lemons were separately treated as described in Part 7 using the compounds and under the conditions in Table 6. The results of the different treatments are shown in Tables 6 and 7 below.

TABLE 6
Effect of Various Treatments on Decay Caused by Sour Rot
First Set of Data

| | Mean |
|---|---|
| Treatment | Decay (%) |
| Sodium bicarbonate 3% wt/vol., rinse, 200 ppm Oxine, 80° F. | 5.768 |
| Soda ash 3% wt/vol, 90° F. | 10.638 |
| Ozine 200 ppm, alone 80° F. | 10.818 |
| Lime sulfur 3% wt/vol 60° F. | 18.028 |
| NaOCl 200 ppm pH 7.5, alone 80° F. | 18.112 |
| Soda ash 3% wt/vol, rinse, 3-8 ppm Oxine 80° F. | 18.349 |
| Dequest 2016D Phosphonic acid 1.0% wt/vol, pH 12, no rinse | 18.528 |
| Oxine 200 ppm & 200 ppm NaOCl pH 7.5 80° F. | 20.220 |
| Oxine 3-8 ppm, 80° F. | 21.710 |
| Oxine 75 ppm, 80° F. | 22.338 |
| Oxine 2-6 ppm, 200 ppm NaOCl, pH 7.5 80° F. | 25.250 |
| Water 80° F., Geotrichum Candidum Inoculum (Control) | 27.207 |
| Inoculate (Control) | 20.395 |

The results presented in Table 6 demonstrate the effectiveness of the method of the present invention in reducing decay due to sour rot in lemons. The decay shown in the lemons treated using an embodiment of the method of the present invention had roughly half the decay rate of the next most effective treatment.

TABLE 7
Effect of Various Treatments on Decay Caused by Sour Rot
Second Set of Data

| | Mean |
|---|---|
| Treatment | Decay (%) |
| Sodium bicarbonate 3% wt/vol rinse, 200 ppm Oxine 80° F. | 5.768 |
| Soda Ash 3% wt/vol 80° F. | 10.856 |
| Oxine 200 ppm 80° F. | 10.878 |
| Lime sulfur 0% wt/vol 80° F. | 16.028 |
| NaOCl 200 ppm pH 7.5, Oxine 80° F. | 18.112 |
| Soda ash 3% w/vol rinse, 28 ppm Oxine 80° F. | 18.528 |
| Dequest 2016D Prosphoric acid 1.0% w/v, pH 12 (No rinse) | 18.628 |
| Oxine 200 ppm & 200 ppm NaOCl pH 7.5 80° F. | 20.220 |
| Oxine 5-8 ppm 80° F. | 21.710 |
| Oxine 75 ppm 80° F. | 22.358 |
| Oxine 3-6 ppm & 200 ppm NaOCl pH 7.5 80° | 25.256 |
| Water 80° F. Inoculate (Control) | 27.267 |
| Inoculate (Control) | 20.596 |

A relatively high concentration of bicarbonate (3%) was used in the test and it is shown to be highly effective for decay control, leading to only 5.7% decay.

9. Effect of Pressure Washing vs. Dipping and Bicarbonate Concentration On Decay Control

The treatments listed below were used on four groups of 25 oranges and four groups of 25 lemons each. All treatments were applied for 35 seconds per group. Treatments 4 through 7 are embodiments of the method of the present invention.

1) Untreated (Control)

2) Low pressure wash with water

3) High pressure wash with water

4) Low pressure wash with 0.5% sodium bicarbonate

5) High pressure, wash with 0.5% sodium bicarbonate

6) Low pressure wash with 1% sodium bicarbonate

7) High Pressure wash with 1% sodium bicarbonate

8) Dipping in 0.5% sodium bicarbonate

9) Dipping in 1% sodium bicarbonate

10) Dip fruit in 3% soda ash

After the treatments are applied, the fruits were stored at 50° F. for 2 weeks and then observed for decay. The results are shown in Table 8 below:

TABLE 8
Effect of Pressure Washing vs. Dipping and Bicarbonate
Concentration On Decay Control

| | Decay (%) | |
|---|---|---|
| Treatment | Oranges | Lemons |
| No pressure washing, 3% Soda ash dip (#10) | 13.0 | 6.8 |
| No pressure washing, 1% sodium bicarbonate dip (#9) | 41.6 | 11.5 |
| Low pressure washing, 1% sodium bicarbonate (#6) | 44.1 | 18.8 |
| High pressure washing, 1% sodium bicarbonate (#7) | 44.9 | 21.9 |
| No pressure washing, 0.5% sodium bicarbonate (#8) | 56.9 | 45.1 |
| High pressure washing, 0.5% sodium bicarbonate (#5) | 76.3 | 55.3 |
| Low pressure washing, 0.5% sodium bicarbonate (#4) | 82.7 | 66.2 |
| Low pressure washing with water only (#2) | 93.6 | 78.6 |
| High pressure washing with water only (#3) | 97.6 | 88.0 |
| Untreated control (#1) | 98.5 | 95.0 |

Means separated by Fisher's protected LSD at P ≦ 0.05 applied to arcs in transformed data.

The lowest rate of decay was in the produce treated using a soda ash dip. However, washing with sodium bicarbonate was consistently superior to washing with water only. The pressure washing treatment was observed to be at least as effective as the tank dipping procedure. Most treatments seem to work better on lemons than oranges, including bicarbonate and soda ash. The pressure washer with chlorinated water alone did not reduce green mold on oranges significantly, but it did slightly and significantly reduce green mold on lemons. Switching from high to low pressure for a given treatment did not seem to affect the decay control significantly.

10. Effect of SOPP, Sodium Bicarbonate & Chlorine Treatment on Navel Orange Decay Control

The decay control efficacy of the following treatments was tested on navel oranges inoculated with P. digitatum spores as described above:

1) Inoculated (Control) with no other treatment.

2) 3% sodium bicarbonate & 200 ppm chlorine in a 35 second dip

3) 3% sodium bicarbonate & 200 ppm chlorine in a pressure washer (100 psi)

4) 200 ppm chlorine alone in a pressure washer (100 psi)

5) 2% solution of SOPP on brushes.

The treatment solution conditions were as follows: sodium bicarbonate concentration of 3%; chlorine concentration of 200-250 ppm; SOPP concentration of 1.6%; pH of 11.3; and, temperature of 70° F.

Navel oranges were collected from field bins and inoculated around mid-afternoon with a 10⁶ spores/ml solution of P. digitatum (M6R strain), by means of a 3 mm inoculating tool. They were randomized into five treatments of four replications per treatment containing about 100 fruit per replication. One treatment was set aside as a control. The next day, the treatments were processed in the following manner. The pressure washer tank was charged with 200 ppm chlorine. The first treatment was dumped onto the roll elevator before the pressure washer and collected from the belt afterwards. In preparation for the second treatment, a 2% SOPP spray was applied to the first few brushes before the pressure washer with a backpack sprayer. The second treatment was then dumped onto the roll elevator, sprayed with SOPP before entering the pressure washer and collected from the belt after the washer. The pressure washer tank was then charged with a 3% solution of sodium bicarbonate while maintaining a level of 200 ppm chlorine. The brushes at the beginning of the washer were rinsed to eliminate the SOPP. The third treatment was then introduced onto the roll elevator before the pressure washer and collected from the belt afterwards. The fruit from the fourth treatment were placed in mesh bags and dipped for 35 seconds in the pressure washer tank. They were not rinsed. All pressure washer treatments were made at low pressure, or about 90 psi, and samples of each treatment were subjected to OPP residue analysis.

All treated fruits were put into tray packs, and stored at 55° F. and 85% relative humidity for two weeks. They were then evaluated and statistically analyzed for decay using the ANOVA program. The results are shown in Tables 9 and 10 below.

TABLE 9
Raw Data - Effect of SOPP, Sodium Bicarbonate & Chlorine Treatment
on Navel Orange Decay Control

| Treatment - Oranges | # Healthy | # Decay | % Decay |
|---|---|---|---|
| Control-R1 | 3 | 108 | 97.3% |
| Control - R2 | 0 | 111 | 100.0% |
| Control-R3 | 1 | 107 | 99.1% |
| Control - R4 | 1 | 110 | 99.1% |
| Control Mean % | | | 98.9% |
| Sodium bicarbonate/Chlorine 35 | 86 | 19 | 18.1% |
| second dip R1 | | | |
| Sodium bicarbonate/Chlorine 35 | 78 | 27 | 25.7% |
| second dip R2 | | | |
| Sodium bicarbonate/Chlorine 35 | 80 | 26 | 24.5% |
| second dip R3 | | | |
| Sodium bicarbonate/Chlorine 35 | 84 | 20 | 19.21% |
| second dip R4 | | | |
| Sodium bicarbonate/Chlorine 35 | | | 21.9% |
| second dip Mean % | | | |
| Pressure Wash Sodium | 88 | 20 | 18.5% |
| bicarbonate/Chlorine R1 | | | |
| Pressure Wash Sodium | 84 | 23 | 21.5% |
| bicarbonate/Chlorine R2 | | | |
| Pressure Wash Sodium | 89 | 16 | 15.2% |
| bicarbonate/Chlorine R3 | | | |
| Pressure Wash Sodium | 78 | 27 | 25.7% |
| bicarbonate/Chlorine R4 | | | |
| Pressure Wash Sodium | | | 20.2% |
| bicarbonate/Chlorine Mean % | | | |
| Chlorine @ 200 ppm R1 | 0 | 105 | 100.0% |
| Chlorine @ 200 ppm R2 | 1 | 104 | 99.0% |
| Chlorine @ 200 ppm R3 | 1 | 95 | 99.0% |
| Chlorine @ 200 ppm R4 | 1 | 98 | 99.0% |
| Chlorine @ 200 ppm Mean % | | | 99.3% |
| OPP @ 1.6% R1 | 7 | 98 | 93.3% |
| OPP @ 1.6% R2 | 4 | 100 | 96.2% |
| OPP @ 1.6% R3 | 5 | 100 | 95.2% |
| OPP @ 1.6% R4 | 8 | 97 | 92.4% |
| OPP @ 1.6% Mean % | | | 94.3% |

The R1 through R4 notation denotes replicates. The raw data is shown in Table 9 above and the averaged values are in Table 10 below. The averaged values also are shown in FIG. 1 in order of ascending decay control; that is (a) represents inoculated control, (b) represents wash with chlorine only, (c) represents wash with OPP spray, (d) represents immersion without pressure in sodium bicarbonate and chlorine, and (e) represents pressure washing in sodium bicarbonate and chlorine.

TABLE 10
Effect of SOPP, Sodium bicarbonate & Chlorine on Navel Oranges
Decay Control

| | | Decay | |
|---|---|---|---|
| | Mean Decay | Control | OPP |
| Treatment | (%) | (%) | Residue |
| 1-Inoculated Control (No other | 98.9% (a)* | 0.0% | <0.05 ppm |
| treatment) | | | |
| 4-200 ppm Chlorine 100 psi wash | 99.3% (a) | 0.0% | <0.05 ppm |
| 5-1.6% OPP spray, 200 ppm Cl, 100 | 94.3% (a) | 4.5% | 0.23 ppm |
| psi wash | | | |
| 2-3% Sodium bicarbonate/200 ppm | 21.9% (b) | 77% | 0.19 ppm |
| Cl 35 sec dip | | | |
| 3-3% Sodium bicarbonate/200 ppm | 20.2% (b) | 78.7% | 0.06 ppm |
| Cl 100 psi wash | | | |

*Values with unlike letters are significantly different at a 95% confidence level.

The most effective treatments at significantly equal rates were the sodium bicarbonate plus chlorine treatments. The treatments encompassing OPP and chlorine alone were ineffective and not significantly different from the inoculated control.

This method of OPP application was more thorough than previously done. One of the best embodiments of the method was utilized for this test, a non-recovery spray bar over the roll elevator, to apply OPP. With the backpack sprayer, twice as much volume was applied over a larger, more comprehensive area than with a spray bar. The OPP treatment produced fairly low residues on the fruit. In general, any application is more effective with the use of more brushes with SOPP before the high pressure washer. The original solution strength was only 1.6% SOPP instead of the 2% targeted. The pH appeared low at 11.3, but was measured only by a digital pH meter; at such high pH, paper strips are often more accurate than electrodes for pH measurements. The OPP residue in the sodium bicarbonate/chlorine dip treatment was surprisingly high.

11. Effect on Fruit Decay of Borax/Boric Acid, Sodium Bicarbonate & Soda Ash Treatment

This test compared the effectiveness of use of sodium bicarbonate, borax/boric acid and soda ash on the decay of oranges and lemons. The treatment solution conditions were identical to those described in Part 10. The fruits were randomly allocated into several groups as described above, and treated as follows. Soda ash was added to a tank where the fruits were dipped, as was one of the bicarbonate groups. A second group of fruit was pressure washed with sodium bicarbonate, and another group with Borax/Boric Acid. The latter treatment was conducted at 200 ppm 2 parts Boric Acid/2 parts Borax to avoid precipitation and maintain its solubility. Control fruit, treated only with water, was also included in the testing.

The results of this testing are shown in FIG. 2. The control samples showed more than 96% decay for oranges and 99% for lemons, whereas all other treatments provided excellent control of fruit decay (less than 5% decay). In particular, the treatments within the scope of the present invention were among those exhibiting high effectiveness.

12. Effect of Various Treatments on Green Mold Decay of Oranges and Lemons

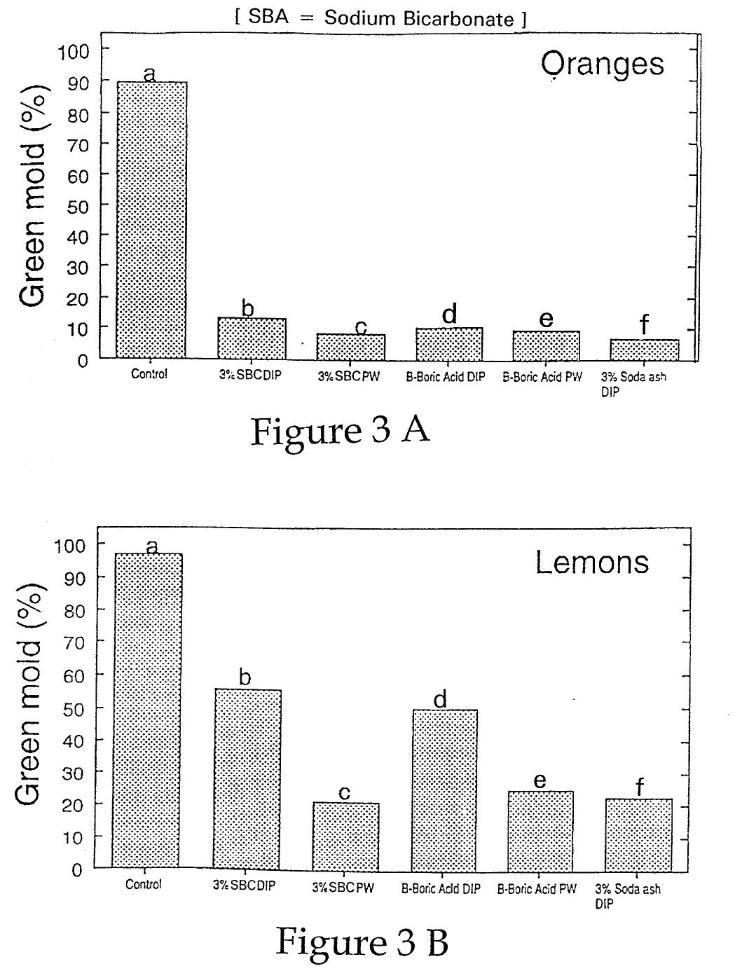

This test assessed the effect of various treatments on 4-5 day old fruit. Randomly selected oranges and lemons, were divided in various groups, and treated with water (control), 3% sodium bicarbonate (dip and pressure washing), borax/boric acid (pressure washing), and 3% soda ash (dip). The results are shown in FIGS. 3A and 3B.

The control samples showed high spoilage (in excess of 90%), whereas all treatments were effective even in 4-5 day old oranges (less than 10% spoilage). In lemons, the treatments were less effective due to the delay in their application during the testing procedure. Treatment using pressure washing with sodium bicarbonate solution was more effective than all but soda ash treatment.

13. Effect of Bicarbonate Concentration on Decay Control

This test evaluated the decay control efficacy on lemons and navel oranges of sodium bicarbonate at concentrations of 1% and 3% in addition to 200 ppm hypochlorite. The treating solution was applied in the pressure washer at low pressure and in the soak tank. Field fresh lemons and non-gassed navel oranges (weak, five day old TI variety citrus fruit) were used in this test and evaluated for inoculated (P. digitatum) and sterile punctured treatments.

The various groups of fruit were treated with 1% or 3% sodium bicarbonate and 200 ppm hypochlorite in a pressure washer or tank (dip), and the efficacy of inoculated and non-inoculated, punctured lemons and oranges were tested. Fresh field lemons and five day old TI's exhibiting significant of puff were collected. Each variety was randomized into six treatments (twelve total) containing four replications of about fifty fruit per replication. The treatments other than the controls were individually marked around the diameters for identification purposes. The fruit in one lemon and one orange group were inoculated 3 mm deep with a 106 spores/ml solution of P. digitatum, M6R strain, and set aside as controls. Two lemon and two orange treatments were also inoculated and set aside for approximately 18 hours. The following day, the fruit in one lemon and one orange treatment were punctured with a sterile 3 mm inoculating tool and set aside as controls. The remaining two lemon treatments and two orange treatments were punctured with a sterile 3 mm inoculating tool. The soak tank and washer solution were charged at 1% sodium bicarbonate and 200 ppm chlorine. Half of the treatments were introduced at the elevator just before the washer and retrieved from the elevator just after the soak tank. The fruit were not rinsed. The soak tank and washer solution were charged at 3% sodium bicarbonate and 200 ppm chlorine. The second half of the treatments were treated exactly as per above in the 1% solution. All treatments were made within about a two hour time frame along with the house fruit. Spore samples were taken and prepared by throughout the duration of the treatments. All twelve treatments were stored at 55° F., and analyzed for decay two weeks later. The pressure washer and soak tank were tied together so that the water solution circulated through both units. The results of this testing are shown in Table 11 below.

TABLE 11
Effect of Bicarbonate Concentration on Orange and Lemon Decay

| Lemons | | Oranges | |
|---|---|---|---|
| | Mean | | Mean |
| Treatment | Decay % | Treatment | Decay % |
| Inoculated (Control) | 94.3 a* | Inoculated Control | 99.5 a |
| Inoculated 1% Sodium | 11.9 b | Inoculated 1% Sodium | 72.7 b |
| bicarbonate/Cl | | bicarbonate/Cl | |
| Inoculated 3% Sodium | 4.8 c | Inoculated 3% Sodium | 23.5 c |
| bicarbonate/Cl | | bicarbonate/Cl | |
| Punctured (Control) | 7.8 a | Punctured Control | 28.0 b |
| Punctured 1% Sodium | 2.0 b | Punctured 1% Sodium | 50.0 a |
| bicarbonate/Cl | | bicarbonate/Cl | |
| Punctured 3% Sodium | 0.0 b | Punctured 3% Sodium | 24.5 b |
| bicarbonate/Cl | | bicarbonate/Cl | |

*Values followed by unlike letters are significantly different and ranked alphabetically.
The ANOVA statistics program was run for all four series of treatments.

The oranges decayed at a higher rate than the lemons, possibly because of their weakened original condition. Both the dip and pressure washing sodium bicarbonate/200 ppm chlorine treatments improved decay control. The 3% sodium bicarbonate/chlorine solution was almost always significantly better than the 1% solution on both lemons and oranges at a ratio of about two to one or more.

In several samples taken from the tank solution throughout the test, relatively low numbers of Penicillium spores were found, as may be seen on Table 12 below.

TABLE 12
Penicillium Spore Colony Counts

| | |
|---|---|
| Sample Sodium bicarbonate | |
| Conc. | Live Spores/ml |

| | | |
|---|---|---|
| 1. | 1% | 2900 |
| 2. | 1% | 100 |
| 3. | 1% | 450 |
| 4. | 1% | 100 |
| 5. | 3% | 50 |
| 6. | 3% | 0 |
| 7. | 3% | 0 |
| 8. | 3% | 50 |

The raw data for oranges are shown in Table 13, below, and for lemons in Table 14, below.

TABLE 13
Comparative Effect of Various Concentrations
of Bicarbonate on Decay of Navel Changes

| | | | | | Mean |
|---|---|---|---|---|---|
| | | No. | | | Decay |
| Treatment | No. | Decay | #Healthy | % Decay | (%) |
| Inoculated Control R1 | 46 | 45 | 1 | 97.8% | 99.5 a* |
| Inoculated Control R2 | 46 | 46 | 0 | 100.0% | |
| Inoculated Control R3 | 47 | 47 | 0 | 100.0% | |
| Inoculated Control R4 | 47 | 47 | 0 | 100.0% | |
| Inoculated 1% Sodium | 43 | 31 | 12 | 72.1% | 72.7 b |
| bicarbonate R1 | | | | | |
| Inoculated 1% Sodium | 43 | 32 | 11 | 74.4% | |
| bicarbonate R2 | | | | | |
| Inoculated 1% Sodium | 43 | 32 | 11 | 74.4% | |
| bicarbonate R3 | | | | | |
| Inoculated 1% Sodium | 43 | 30 | 13 | 69.8% | |
| bicarbonate R4 | | | | | |
| Inoculated 3% Sodium | 44 | 9 | 35 | 20.5% | 23.5 c |
| bicarbonate R1 | | | | | |
| Inoculated 3% Sodium | 44 | 9 | 35 | 20.5% | |
| bicarbonate R2 | | | | | |
| Inoculated 3% Sodium | 44 | 10 | 34 | 22.7% | |
| bicarbonate R3 | | | | | |
| Inoculated 3% Sodium | 43 | 13 | 30 | 30.2% | |
| bicarbonate R4 | | | | | |
| Punctured Control R1 | 44 | 10 | 34 | 22.7% | 28.0 b |
| Punctured Control R2 | 44 | 14 | 30 | 31.8% | |
| Punctured Control R3 | 44 | 12 | 32 | 27.3% | |
| Punctured Control R4 | 43 | 13 | 30 | 30.2% | |
| Punctured 1% Sodium | 38 | 18 | 20 | 47.4% | 50.0 a |
| bicarbonate R1 | | | | | |
| Punctured 1% Sodium | 36 | 21 | 15 | 58.3% | |
| bicarbonate R2 | | | | | |
| Punctured 1% Sodium | 36 | 19 | 17 | 52.8% | |
| bicarbonate R3 | | | | | |
| Punctured 1% Sodium | 36 | 15 | 21 | 41.7% | |
| bicarbonate R4 | | | | | |
| Punctured 3% Sodium | 46 | 11 | 35 | 23.9% | 24.5 b |
| bicarbonate R1 | | | | | |
| Punctured 3% Sodium | 46 | 10 | 36 | 21.7% | |
| bicarbonate R2 | | | | | |
| Punctured 3% Sodium | 46 | 8 | 38 | 17.4% | |
| bicarbonate R3 | | | | | |
| Punctured 3% Sodium | 46 | 16 | 30 | 34.8% | |
| bicarbonate R4 | | | | | |

*Values followed by unlike letters are significantly different and ranked alphabetically. The ANOVA program was run separately for the punctured and inoculated series of treatments.

TABLE 14
Comparative Effect of Various Concentrations
of Bicarbonate on Lemon Decay

| | | | | | Mean |
|---|---|---|---|---|---|
| | | No. | | | Decay |
| Treatment | No. | Decay | #Healthy | % Decay | (%) |
| Inoculated Control R1 | 48 | 46 | 2 | 95.8% | 94.3 a |
| Inoculated Control R2 | 48 | 45 | 3 | 93.8% | |
| Inoculated Control R3 | 48 | 47 | 1 | 89.6% | |
| Inoculated Control R4 | 47 | 47 | 0 | 100.0% | |
| Inoculated 1% BC R1 | 64 | 9 | 55 | 14.1% | 11.9 b |
| Inoculated 1% BC R2 | 63 | 8 | 55 | 12.7% | |
| Inoculated 1% BC R3 | 63 | 7 | 56 | 11.1% | |
| Inoculated 1% BC R4 | 62 | 6 | 56 | 9.7% | |
| Inoculated 3% BC R1 | 51 | 2 | 49 | 3.9% | 4.8 c |
| Inoculated 3% BC R2 | 51 | 2 | 49 | 3.9% | |
| Inoculated 3% BC R3 | 52 | 3 | 49 | 5.8% | |
| Inoculated 3% BC R4 | 52 | 3 | 49 | 5.8% | |
| Punctured Control R1 | 48 | 6 | 42 | 12.5% | 7.8 a |
| Punctured Control R2 | 48 | 0 | 48 | 0.0% | |
| Punctured Control R3 | 48 | 5 | 43 | 10.4% | |
| Punctured Control R4 | 48 | 4 | 44 | 8.3% | |
| Punctured 1% BC R1 | 64 | 0 | 64 | 0.0 | 2.0 b |
| Punctured 1% BC R2 | 64 | 2 | 62 | 3.1% | |
| Punctured 1% BC R3 | 64 | 2 | 62 | 3.1% | |
| Punctured 1% BC R4 | 64 | 1 | 63 | 1.6% | |
| Punctured 3% BC R1 | 34 | 0 | 64 | 0.0% | 0.0 b |
| Punctured 3% BC R2 | 34 | 0 | 64 | 0.0% | |
| Punctured 3% BC R3 | 34 | 0 | 64 | 0.0% | |
| Punctured 3% BC R4 | 34 | 0 | 64 | 0.0% | |

*Values followed by unlike letters are significantly different and ranked alphabetically. The ANOVA program was run separately for the punctured and inoculated series of treatments.

This experiment confirms that weak fruit tend to be more susceptible to decay, regardless of variety. Treatment with 3% sodium bicarbonate was shown to be superior to that with 1% sodium bicarbonate. Addition of 200 ppm chlorine into the treatment solution also appears to be highly effective in reducing Penicillium spores in the treated fruit.

14. Effect on Spore Count on Treatment With Different Bicarbonate Concentrations and HOCl

Freshly wounded, non-inoculated lemons and oranges were immersed in a solution of 1% or 3% sodium bicarbonate also including 200 ppm free HOCl. Commercial oranges were running through the tank during the treatments, and the tank temperature was about 68-75° F. 40 ml samples were taken by sampling small volumes over the length of the tank. They were mixed, and then dispensed into a sterile Corning tube containing 1 ml of 1000 ppm calcium thiosulfate. The samples were iced, 0.2 ml were plated on DRBC after a 10×dilution in water, and the number of spores counted. The results are shown in Table 15 below.

TABLE 15
Spore Count After Various Treatments

| | Colonies on | Live | |
|---|---|---|---|
| Sample | 50x dil plate | spores/ml | Tank Conditions/Comments |
| 1 | 58 | 2900 | 1% Sodium bicarbonate/HOCl initial |
| | | | sample, many fruit in tank Line had |
| | | | been running ca. 1 hour w/Sodium |
| | | | bicarbonate/HO |
| 2 | 2 | 100 | 1% Sodium bicarbonate/HOCl about |
| | | | 5 Min later, test fruit running in |
| | | | large numbers |
| 3 | 9 | 450 | 1% Sodium bicarbonate/HOCl about |
| | | | 25 min later, test fruit as before, |
| | | | now running for 30 min |
| 4 | 2 | 100 | 1% Sodium bicarbonate/HOCl |
| | | | another 10 min later, test fruit |
| | | | now running for 40 min |
| 5 | 1 | 50 | 3% Sodium bicarbonate/HOCl initial |
| | | | sample in 3% Sodium bicarbonate |
| | | | test |
| | | | About 1 hr passed since 1% Sodium |
| | | | bicarbonate in use, a few fruit just |
| | | | beginning to run again |
| 6 | 0 | 0 | 3% Sodium bicarbonate/HOCl second |
| | | | sample in 3% Sodium bicarbonate |
| | | | test |
| | | | About 10 min into test, with |
| | | | commercial fruit running but far |
| | | | fewer than before |
| 7 | 0 | 0 | 3% Sodium bicarbonate/HOCl after |
| | | | break of 20 min or so About 30 min |
| | | | into test, With commercial fruit just |
| | | | beginning to run when sample taken |
| 8 | 1 | 50 | 3% Sodium bicarbonate/HOCl end of |
| | | | the test sample About 45 min into |
| | | | test, with commercial fruit running, |
| | | | but far fewer than in 1% Sodium |
| | | | bicarbonate test |

The results shown in the above table indicate that viable spores did not accumulate in the water during the test, and they were always very low. Breaks or pauses, such as between the 1% and 3% sodium bicarbonate tests, allowed the HOCl time to kill most all the spores in the tank. Because fewer fruit were running and for less time, the spore numbers were always very low during the 3% sodium bicarbonate treatment.

16. Effect of Post-harvest Bicarbonate High Pressure Washing to Control Green Mold

In this example, the fruit were wound-inoculated with P. digitatum spores, and 24 hours later treated with sodium bicarbonate (1 or 3% wt/vol) at 18° C. applied for 35 seconds in a tank or by HPSW at 2150 kPa (320 psi). P. digitarum (Pers.:Fr.) isolate M6R, was cultured 1 to 2 weeks on potato dextrose agar. Spores were harvested by adding 5 ml of water containing 0.05% Triton X-100 to the Petri dish, rubbing the surface with a sterile glass rod, passing the suspension through two layers of cheese cloth. The suspension was diluted with water to an absorbance of 0.1 at 425 nm determined with a spectrophotometer; this density contains about 10⁴ spores per ml.

Lemons and oranges that had been commercially harvested no more than two days before use, were randomized and inoculated with P. digitarum 24 (±2) hours before treatment. The inoculation method employed simulates infections that occur under commercial conditions and it has been recommended for determining the effectiveness of fungicides. Fruit were inoculated by immersing a stainless steel rod with a probe tip 2 mm long and 1 mm wide into the spore suspension and wounding each fruit once. The temperature of the fruit at the time of inoculation was 20° C.±1° C. After all treatments were applied, the fruit were placed into plastic cavity trays that prevented contact infections and stored at 10° C., a comma storage temperature for citrus fruit, for two weeks when an incidence of green mold was determined. In every test, controls included fruit that were inoculated and not otherwise treated.

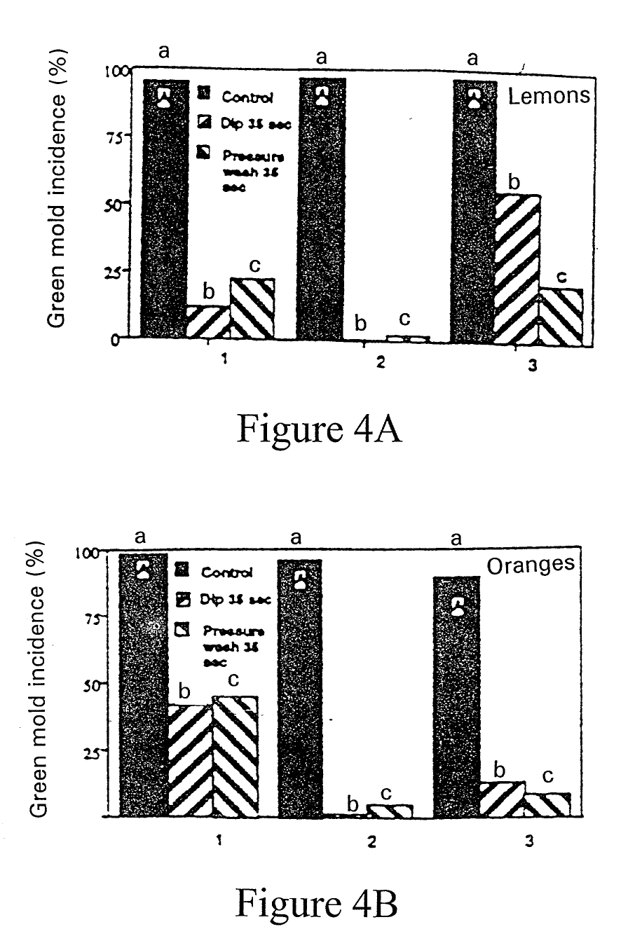

Sodium bicarbonate was applied either at 18° C. for 35 seconds in a tank or by HPSW at 2150 kPa (320 psi), and the results were compared. The pH of the Sodium bicarbonate solution was 8.3 and it contained 200 micrograms/ml free NaOCl, which was measured colorimetrically by the DPD method with a Hach DR 890 calorimeter. The sodium bicarbonate concentration was 1, 3, and 3% (wt/vol) in tests 1, 2, and 3, respectively. The temperature of solution at the time of treatment was 25° C.±3° C. The concentration of the solution was monitored periodically with a salt refractometer and did not change during the tests. All tests were conducted with 6 replicates of 60 oranges and 75 lemons, each. The results of this test are shown in FIGS. 4A and 4B.

None of the fruit were injured by any of the treatments applied. Among oranges, HPSW or tank treatment were equally effective in all tests. Among lemons, the HPSW was slightly inferior, equal, and superior to tank treatment in tests 1, 2, and 3, respectively. The HPSW with sodium bicarbonate treatments reduced decay from 97% among controls to a mean of 17 and 20%, respectively, for lemons and oranges. The sodium bicarbonate tank treatment reduced the incidence of decay from 97% (Controls) to a mean of 22 and 19%, respectively, for lemons and oranges.

17. Effect of Various Concentrations of HOCl Solutions Without Use of Sodium Bicarbonate

This testing was similar to that described in Part 4 above in that it uses only HOCl without sodium bicarbonate in the solution. The concentrations of HOCl used were (a) 400 psi pressure, no chlorine, (b) 400 psi pressure, 75-150 ppm chlorine, (c) High pressure, no chlorine, and (d) High pressure, high chlorine.

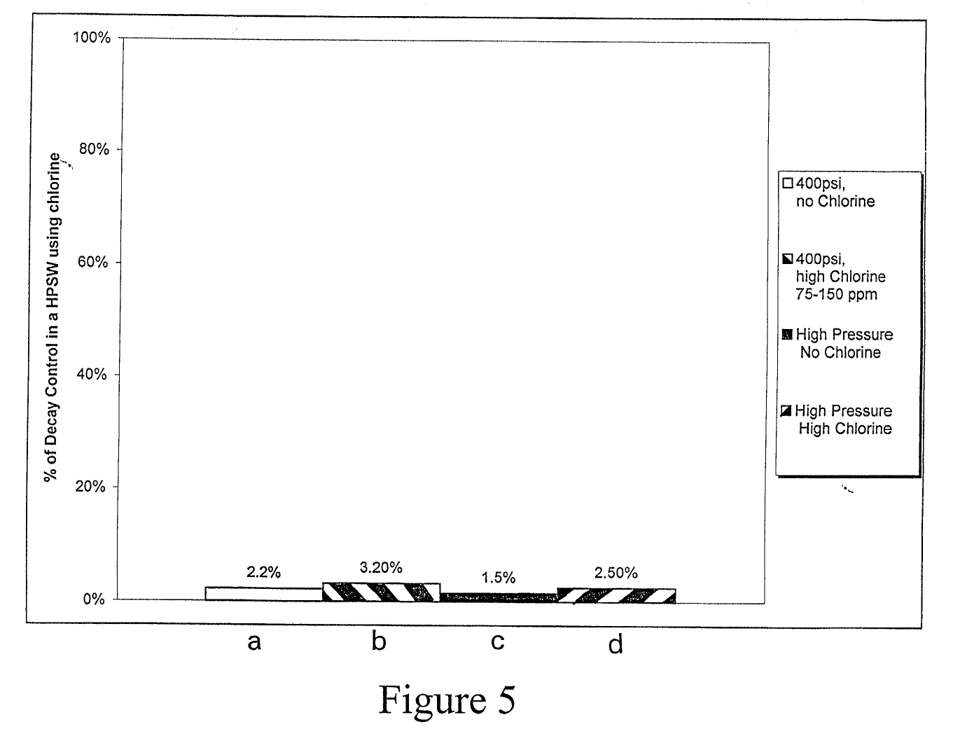

The results of the testing are shown in FIG. 5. None of the treatments appeared to reduce the decay of the fruit substantially beyond the control fruit.

18. Effects of Varying Bicarbonate Concentration

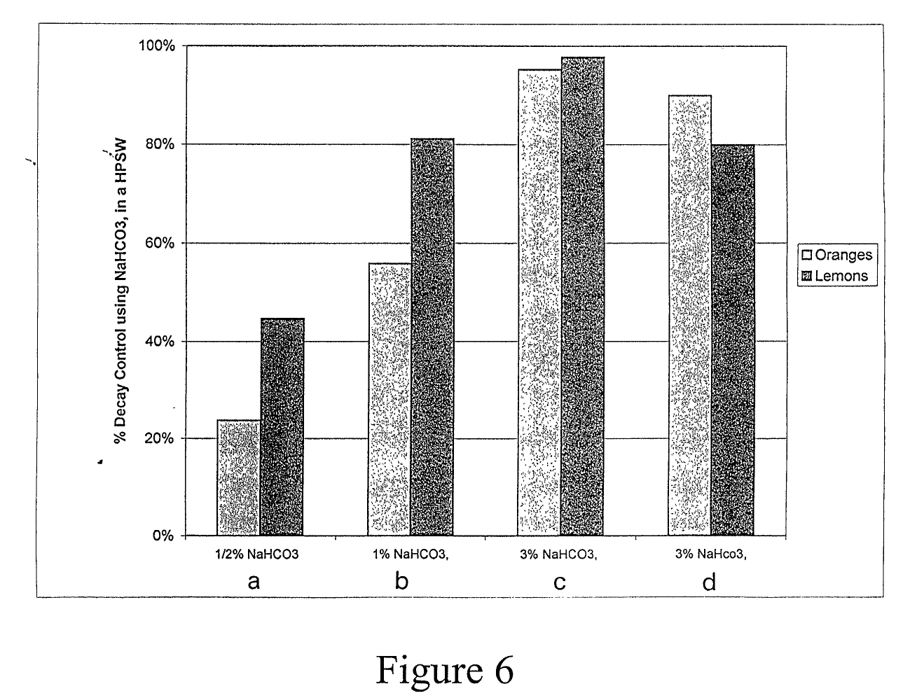

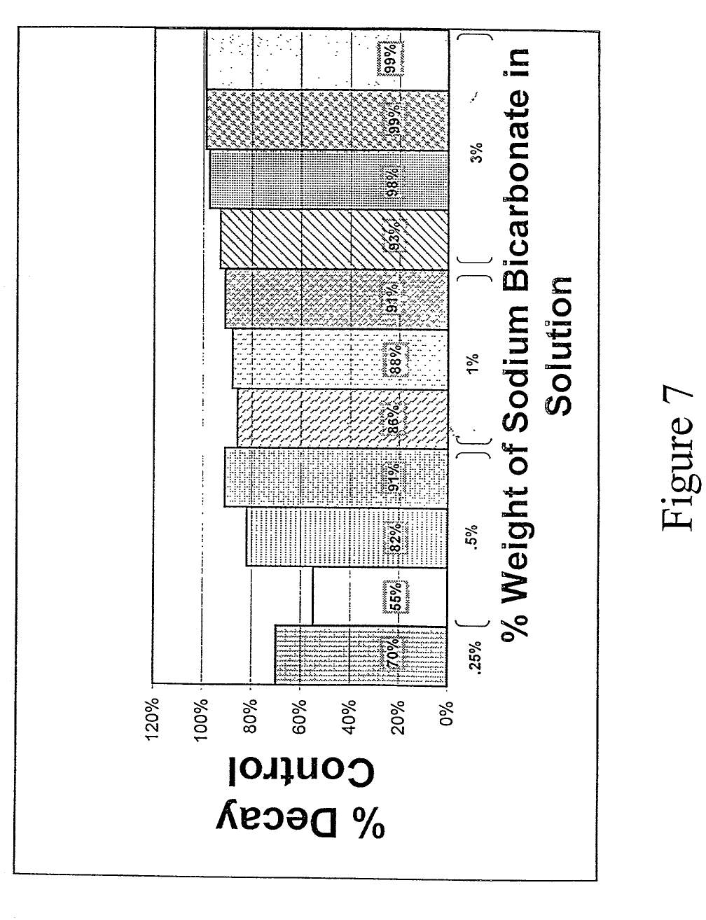

This testing demonstrated the effect of varying the bicarbonate concentration in the washing solution. Concentrations of 0.5%, 1% and 3% sodium bicarbonate were tested, and the results of the testing are shown in FIG. 6. In all cases when the fruits were fresh, the higher concentration of the agent provided the highest decay control (3% bicarbonate, close to 100% control), except in the case of 3-day-old lemons (d). A second experiment also employing 0.25% (w/v) sodium bicarbonate supported these findings. The results for this are shown in FIG. 7.

### REFERENCES

The following references are cited for enablement purposes and, therefore, their relevant portions are incorporated herein by reference.

Barger, W. R. 1928. Sodium bicarbonate as a citrus fruit disinfectant. California Citrograph 13:164-174.

Eckert, L. W., and Brown, G. E. 1986. Evaluation of postharvest treatments for citrus fruits. Pages 92-97 in: Methods for Evaluating Pesticides for Control of Plant Pathogens. K. D. Hickey, ed. American Phytopathological Society, St. Paul, Minn.

Pelser, P. du T. 1974. Recommendations for the control of post-harvest decay of citrus fruits. S. A. Co-operative Citrus Exchange Ltd.

Peter D. Petracek, D. Frank Kelsey, and Craig Davis. 1998. Response of citrus fruit to high-pressure washing. 123(4):661-667.

Smilanick, J. L., Mackey, B. E., Reese, R., Usall, J., and Margosan, D. A. 1997. Influence of the concentration of soda ash, temperature, and immersion period on the control of postharvest green mold of oranges. Plant Dis. 81:379-382.

Smilanick, J. L., Margosan, D. A., and Henson, D. J. 1995. Evaluation of heated solutions of sulfur dioxide, ethanol, and hydrogen peroxide to control postharvest green mold of lemons. Plant Dis. 79:742-747.

Smilanick, J. L., Margosan, D. A., Mlikota, F., Usall, J., and Michael, 1. 1999. Control of citrus green mold by carbonate and bicarbonate salts and the influence of commercial post-harvest practices on their efficacy. Plant Disease 83:139-145.

Usall, J., Pons, J., Palou, L., Vifias, I., and Smilanick, J. L. 1999. Alternatives a los productos quimicos de sintesis en post-cosecha de citricos en Espana y EEUU. Phytoma 110:58-64.

Walker, G. P., Morse, J. G., and Arpaia, M. L. 1996. Evaluation of a high-pressure washer for post-harvest removal of California red scale from citrus fruit. J. Economic Entomology 89 (1) p. 148-155.

Bull, C. T., Stack, J. P., and Smilanick, J. L. 1997. Pseudomonas Syingae strains ESC-10 and ESC-11 survive in wounds on citrus and control green and blue mold of citrus. Biological control 8:18-88.

Eckert, J. W. 1995 Post-harvest Diseases control experience with citrus fruits. Tree Fruit post-harvest Journal 6:9-12.

Eckert, J. W., and Brown, G. E. 1986. Evaluation of post-harvest treatments for citrus fruits. Pages 92-97 in; Methods for Evaluating Pesticides for Control of Plant Pathogens. K. D. Hickey, ed. American Phytopathological Society, St. Paul, Minn.

Eckert, J. W., Sievert. J. R., and Ratnayake, M. 1994. Reduction of imazalil effectiveness against citrus green mold in California packing houses by resistant biotypes of Penicillium digitalum. Plant Dis. 78:971-974.

Houck, L. G. 1967. Hot water treatments for control of penicillium digitatum green mold of Eureka lemons. (Abstr.) Phytopathology 57:99.

McDowell, D. K., and Smilanick, J. L. 1995. Post-harvest biological control of green mold on lemon fruit with Pseudomonas species. Biological and Cultural Tests for Control of Plant Diseases 10:52.

Smilanick, J. L., Michael, I. F., Mansour, M. F., Mackey, B. E., Margosan, D. A., Flores, D., and Weist, C. F. 1997. Improved control of green mold of citrus with imazalil in warm water compared to its use in wax. Plant Dis. 81:in press.

Smilanick, J. L., Mackey, B. E., Reese, R., Usall, J., and Margosan, D. A., 1997. Influence of concentration of soda ash temperature, and immersion period on the control of postharvest green mold of oranges. Plant Disease 81:379-382.

Wild, B. L., and Hood, C. W. 1989. Hot dip treatments reduce chilling injury in long-term storage of ‘Valencia’ oranges. HortScience 24; 109-110.

The relevant disclosures of all scientific publications and patent references cited in this patent are specifically intended to be incorporated herein by reference, particularly in reference to preparatory methods and technologies which are enabling of the invention.

As demonstrated in the discussion and examples above, the methods of the present invention allow for good decay control of treated produce, with related advantages of ease of use and cost-efficiency. Although the invention has been disclosed in detail with reference only to the preferred method, those skilled in the art will appreciate that additional methods for making alumina are within the scope of the invention. Accordingly, the invention is defined only by the following claims.

I claim:

1. A method for washing produce comprising spraying the produce using a washing solution under pressure for a duration, wherein the washing solution comprises an alkaline salt of bicarbonate and has a temperature and pH effective to inhibit or eliminate decay of the produce.

2. A method as defined in claim 1, wherein the alkaline salt of bicarbonate is sodium bicarbonate.

3. A method as defined in claim 1, wherein the pH of the washing solution is between about 7.0 and about 9.5.

4. A method as defined in claim 3, wherein the pH of the washing solution is between about 8.0 and about 8.4.

5. A method as defined in claim 1, wherein the washing solution comprises a sanitizing agent.

6. A method as defined in claim 5, wherein the sanitizing agent is selected from the group consisting of ClO₂, ozone, and an alkaline salt of hypochlorite.

7. A method as defined in claim 6, wherein the sanitizing agent is sodium hypochlorite.

8. A method as defined in claim 7, wherein the sodium hypochlorite is present in the washing solution at a concentration of about 200 ppm.

9. A method as defined in claim 1, wherein the alkaline salt of bicarbonate is present in an amount between about 0.25% and about 6% in the washing solution.

10. A method as defined in claim 9, wherein the alkaline salt of bicarbonate is present in an amount between about 0.1% and about 5% in the washing solution.

11. A method as defined in claim 10, wherein the alkaline salt of bicarbonate is present in an amount of about 3% in the washing solution.

12. A method as defined in claim 1, wherein the temperature of the washing solution is between about 10° C. and about 40° C.

13. A method as defined in claim 12, wherein the temperature of the washing solution is between about 20° C. and about 30° C.

14. A method as defined in claim 1, wherein the duration of the spraying is between about 1 second and about 10 minutes.

15. A method as defined in claim 14, wherein the duration of the spraying is between about 5 seconds and about 5 minutes.

16. A method as defined in claim 15, wherein the duration of the spraying is between about 15 seconds and about 1 minute.

17. A method as defined in claim 1, further comprising a step of immersing the produce in a second solution comprising an alkaline salt of bicarbonate.

18. A method as defined in claim 1, further comprising a step of rinsing the produce using an aqueous solution after the step of spraying the produce.

19. A method as defined in claim 1, wherein the produce is selected from the group consisting of oranges, lemons, tangerines, tangelos, grape fruits, grapes, bananas, apples, pears, peaches, nectarines, pomegranates, papaya, plums, melons, cucumbers, zucchini, carrots, mushrooms, peppers, broccoli, artichokes, cauliflower, tomatoes, potatoes, squash and celery.

20. A method as defined in claim 1, wherein the pressure of the washing solution is between about 50 and about 500 lbs/in².

21. A method as defined in claim 20, wherein the pressure of the washing solution is between about 60 and about 350 lbs/in².

22. A method for washing produce comprising:
contacting the produce with a washing solution comprising an alkaline salt of bicarbonate and a sanitizing agent at concentrations, for a duration, and at a temperature and pH effective to inhibit or eliminate decay of the produce.

23. A method as defined in claim 22, wherein the step of contacting includes immersing the produce in the washing solution.

24. A method as defined in claim 22, wherein the step of contacting includes spraying the produce using the washing solution.

25. A method as defined in claim 22, wherein the alkaline salt of bicarbonate is sodium bicarbonate.

26. A method as defined in claim 22, wherein the pH of the washing solution is between about 7.0 and about 9.5.

27. A method as defined in claim 26 wherein the pH of the washing solution is between about 8.0 and about 8.4.

28. A method as defined in claim 22, wherein the sanitizing agent is selected from the group consisting of ClO₂, ozone, and an alkaline salt of hypochlorite.

29. A method as defined in claim 28, wherein the sanitizing agent is sodium hypochlorite.

30. A method as defined in claim 29, wherein the sodium hypochlorite is present in the washing solution at a concentration of about 200 ppm.

31. A method as defined in claim 22, wherein the alkaline salt of bicarbonate is present in an amount between about 0.25% and about 6% in the washing solution.

32. A method as defined in claim 31, wherein the alkaline salt of bicarbonate is present in an amount between about 0.1% and about 5% in the washing solution.

33. A method as defined in claim 32, wherein the alkaline salt of bicarbonate is present in an amount of about 3% in the washing solution.

34. A method as defined in claim 22, wherein the temperature of the washing solution is between about 10° C. and about 40° C.

35 A method as defined in claim 34, wherein the temperature of the washing solution is between about 20° C. and about 30° C.

36. A method as defined in claim 22, wherein the step of contacting is conducted for a duration between about 1 second and about 10 minutes.

37. A method as defined in claim 36, wherein the step of contacting is conducted for a duration between about 5 seconds and about 5 minutes.

38. A method as defined in claim 39, wherein the step of contacting is conducted for a duration between about 15 seconds and about 1 minute.

39. A method as defined in 22, further comprising a step of rinsing the produce in an aqueous solution after the step of contacting the produce.

40. An apparatus for use in washing produce, comprising:
an alkaline salt of bicarbonate;
a sanitizing agent; and
instructions for employing the alkaline salt of bicarbonate and sanitizing agent in washing produce under conditions effective in inhibiting or eliminating decay of the produce.

41. The apparatus of claim 40, further comprising a measuring means.

42. The apparatus of claim 40, wherein the alkaline salt of bicarbonate is sodium bicarbonate.

43. The apparatus of claim 40, further comprising an agent for adjusting the pH of a washing solution prepared using the apparatus.

44. The apparatus of claim 40, wherein the sanitizing agent is selected from the group consisting of ClO₂, ozone and an alkaline salt of hypochlorite.
